(19) United States
(12) Patent Application Publication
(10) Pub. No.: US 2023/0294026 A1
(43) Pub. Date: Sep. 21, 2023

(54) ANTI-VIRAL COMPOSITIONS AND METHOD OF KILLING VIRUS

(71) Applicant: BLUACT TECHNOLOGIES GMBH, Opfikon, Zurich (CH)

(72) Inventors: Achana PALIKA, Zurich (CH); Akram RAHIMI, Zurich (CH); Sreenath BOLISETTY, Zurich (CH); Raffaele MEZZENGA, Zurich (CH)

(21) Appl. No.: 18/021,347

(22) PCT Filed: Aug. 13, 2021
(86) PCT No.: PCT/IB2021/057498

§ 371 Date: Feb. 14, 2023

Related U.S. Application Data
(60) Provisional application No. 63/065,824, filed on Aug. 14, 2020.

(51) Int. Cl. B01D 39/20; B01D 39/16

(52) U.S. Cl. CPC B01D 39/2037; B01D 39/1615; B01D 2257/91; B82Y 5/00

(57) ABSTRACT

A method of inactivating a virus that includes the step of contacting a virus with an antiviral composition comprising a hybrid antiviral metal-associated amyloid fibril comprising metal nanoparticles on a surface of an amyloid fibril to contact a virus and an antiviral composition comprising metal nanoparticles on a surface of an amyloid fibril wherein the antiviral composition removes virus and bacterial contaminants in water or air to non-detectable levels when water or air comes into contact with the antiviral composition.

### CROSS-REFERENCE TO RELATED APPLICATION

This application claims priority to and benefit of U.S. Provisional Application Ser. No. 63/065,824, filed on Aug. 14, 2020, entitled “ANTI-VIRAL COMPOSITIONS AND METHODS OF KILLING VIRUS,” the disclosure of which is hereby incorporated herein by reference in its entirety.

### BACKGROUND

Viruses essentially consist of genetic material, a protein capsid and the outer envelopes (in case of enveloped viruses). They cannot live in an inanimate environment but are dependent on favorable conditions in living host cells to replicate. Many viral infections eventually result in the death of the host cell of any host organism, including microorganisms, plants, and animals, including human beings. Viral epidemics for instance caused by smallpox or poliovirus are well documented and history has seen many viral epidemics. The 1918 influenza pandemic is the last great pandemic responsible for the death of several tens of millions of people across the planet. More recent viral epidemics include the AIDS, SARS (2002), Swine flu (2009), Ebola (2013) and the recent outbreak of COVID-19 (2019-2020).

The COVID-19 outbreak began in December 2019 in Wuhan, Hubei province of China. The causative agent, SARS-CoV-2, was sequenced and isolated by scientists in January 2020. SARS-CoV-2 is associated with the ongoing outbreak of atypical pneumonia (COVID-2019) that has affected millions of people and killed several hundred thousands of those affected in 213 countries as of May 2020. On Jan. 30, 2020, the World Health Organization declared the SARS-CoV-2 epidemic a public health emergency of international concern. On Feb. 11, 2020, the WHO Director-General, Dr. Tedros Adhanom Ghebreyesus, announced that the disease caused by this new CoV was a “COVID-19,” which is the acronym of “coronavirus disease 2019”.

At present, no effective antiviral treatment or vaccine is available for COVID-19. However, a randomized controlled clinical trial is currently underway to assess the efficacy and safety of several antiviral drugs in patients with COVID-19. First-line treatment for fevers includes antipyretic therapy such as paracetamol. Expectorants such as guaifenesin may be used for a cough. The best procedures to control the source of infection are early diagnosis, isolation and supportive treatments. For individuals, good personal hygiene and avoiding crowded places/social distancing will help to prevent COVID-19 infection and other respiratory viral infections.

Most of the current antiviral drugs are small molecules (for example, nucleoside analogues and peptidomimetics), proteins able to stimulate the immune response (for example, interferon), and oligonucleotides (for example, fomivirsen). Favilavir, which is sold under the brand name Avigan®, is an antiviral drug that was approved in Japan in 2014 to treat influenza, and currently also approved for treating COVID-19 in some countries. Some anti-flu drugs such as oseltamivir, laninamivir, peramivir and zanamivir have been applied for the treatment of COVID-19 patients. However, these drugs are not very effective against SARS-Cov-2. Another anti-viral drug, Remdesivir showed efficacy by resisting two viruses similar to Covid-19, SARS-CoV and MERS-CoV, in animals. Clinical trials of Remdesivir, have officially started in a number of hospitals in Wuhan to test its efficacy against COVID-19 and has been shown to have some benefits in shortening the hospitalization time in some patients. In addition, arbidol, an anti-influenza drug targeting the viral hemagglutinin (HA) is being used in a clinical trial against COVID-19. Chloroquine phosphate, an old drug for the treatment of malaria, has been shown to have apparent efficacy and acceptable safety against COVID-19 associated pneumonia.

Since viruses largely depend on the biosynthetic machinery of infected cells for their replication, the specificity of antiviral drugs is far from ideal, resulting in general intrinsic toxicity associated with such treatment. Additionally, many viruses mutate rapidly due to error-prone replication machinery; therefore, they often develop resistance. As discussed above, additionally, numerous antiviral drugs have proven to exhibit toxicity. The antiviral drugs often cannot be active for the broad spectrum of viral infections and it is difficult for the drugs to pass the phase 3 clinical trials.

### SUMMARY

An aspect of the present disclosure is generally directed to a water or air filtration apparatus that includes a support material that holds on or within it an antiviral composition comprising metal nanoparticles on a surface of an amyloid fibril thereby forming a hybrid antiviral metal-associated amyloid fibril.

Another aspect of the present disclosure is generally directed toward an antiviral air or water filtration substrate comprising a material substrate having an antiviral composition engaged with or contained in the material substrate where the antiviral composition consists essentially of iron particles on a surface of an amyloid fibril.

Yet another aspect of the present disclosure is generally directed toward a method of inactivating virus that includes the step of contacting a virus with an antiviral composition comprising causing a hybrid antiviral metal-associated amyloid fibril comprising metal nanoparticles on a surface of an amyloid fibril to contact a virus.

Another aspect of the present disclosure is generally directed to a method of filter virus and other contaminants that includes the steps of: contacting, typically by moving, a fluid containing a virus into contact with a hybrid antiviral metal-associated amyloid fibril comprising metal particles on a surface of an amyloid fibril such that the virus is held by the hybrid antiviral metal-associated amyloid fibril and the virus eliminated from the fluid.

Yet another aspect of the present disclosure is generally directed toward a method of preventing the spread of a virus comprising the step of: either (1) wearing a mask or (2) using an air or water filter to catch virus passing through the mask or the air or water filter wherein the mask or the air or water filter includes a hybrid antiviral metal-associated amyloid fibril.

Another aspect of the present disclosure is generally directed toward a method of preventing infection from a virus that includes the steps of: filtering a fluid through a membrane having a hybrid antiviral metal-associated amyloid fibril comprising iron hydroxide nanoparticles bonded to a surface of an amyloid fibril; and preventing virus from infecting an animal using the membrane having the hybrid antiviral metal-associated amyloid fibril.

Yet another aspect of the present disclosure is generally directed toward a water filter that includes a housing with an interior volume, a water inlet and a water outlet. The interior volume comprises a hybrid antiviral metal-associated amyloid fibril that includes an amyloid fibril containing metal particles in contact with the amyloid fibril and wherein the hybrid antiviral metal-associated amyloid fibril synergistically reduces virus amounts in water passing through the water filter and coming into contact with the hybrid antiviral metal-associated amyloid fibril.

Another aspect of the present disclosure is generally directed toward an air filter that includes an air filtering media having a hybrid antiviral metal-associated amyloid fibril and a membrane or other substrate engaged with the air filtering media.

These and other aspects, objects, and features of the present disclosure and the claimed invention will be understood and appreciated by those skilled in the art upon studying the following specification, claims, and appended drawings.

### BRIEF DESCRIPTION OF THE DRAWINGS

In the drawings:

FIG. 1 is a schematic process for the synthesis of the amyloid-iron hydroxide nanoparticles, which can be used as building blocks for the aerosol membranes.

FIG. 2A is a graphical depiction of the incubation of Phi 6 Bacteriophages with β-lac Amyloid fibrils, Fe nanoparticles and β-lac Amyloid iron hydroxide particle hybrids.

FIG. 2B is a graphical depiction of the incubation of Influenza A virus with β-lac Amyloid fibrils, Fe nanoparticles and β-lac amyloid iron hydroxide particle hybrids.

FIG. 2C is a graphical depiction of the removal of Phi6 bacteriophages with cellulose membranes, β-lac Amyloid fibrils membrane and β-lac amyloid iron hydroxide hybrid membranes.

FIG. 3 is a graphical depiction of the incubation of Phi6 bacteriophage with β-lac Amyloid fibrils and β-lac Amyloid iron hydroxide and iron oxide nanoparticles.

FIG. 4 is a graphical depiction of the incubation of Phi6 bacteriophage with β-lac Amyloid fibrils, and β-lac Amyloid silver particles showing the synergistic effectiveness of the combination of Amyloid fibrils having silver particles thereon in eliminating.

FIG. 5 is a schematic view of a water filter that may incorporate the hybrid antiviral metal-associated amyloid fibrils of the present disclosure.

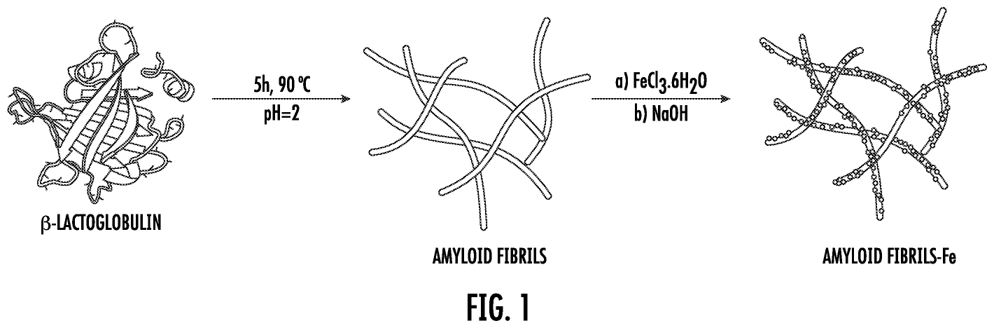

### DETAILED DESCRIPTION

It is to be understood that the disclosure and the claimed invention may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made.

The various embodiments of the anti-viral compositions of the present disclosure, the anti-viral compositions may also be substantially free of any ingredient or feature described herein, provided that the remaining composition still contains all of the required ingredients or features as described herein. In this context, and unless otherwise specified, the term “substantially free” means that the selected composition contains less than a functional amount of the optional ingredient, typically less than 1%, including less than 0.5%, including less than 0.1%, and also including zero percent, by weight of such optional or selected essential ingredient.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the Applicant intends to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.”

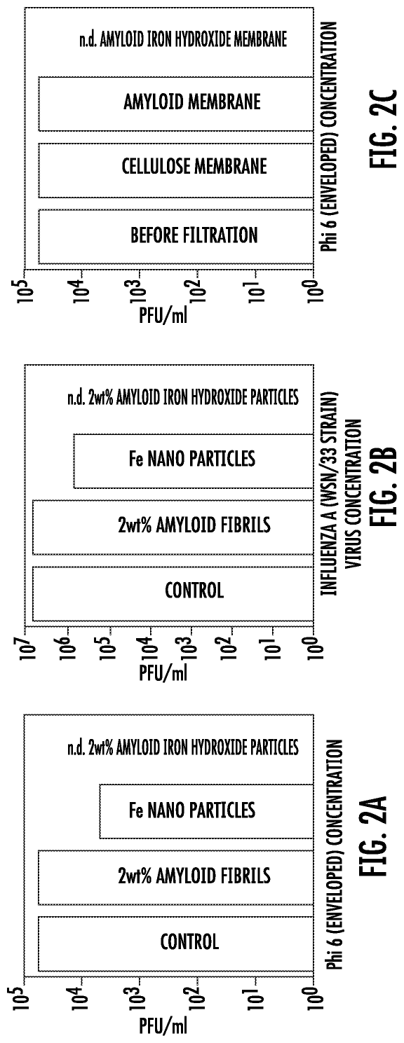

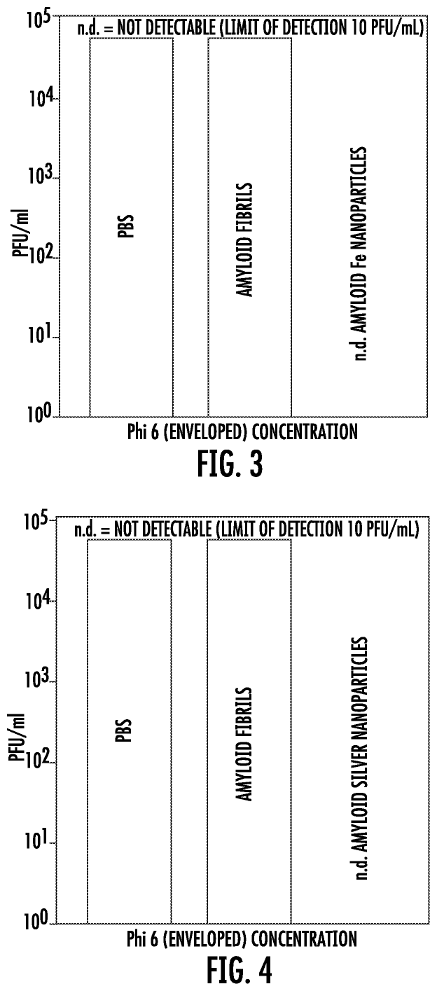

The present disclosure is generally directed to the use of hybrid antiviral metal-associated amyloid fibrils to treat viruses containing air and water. As shown in at least FIGS. 2A-C, FIG. 3 and FIG. 4, the hybrid antiviral metal-associated amyloid fibrils of the present disclosure surprisingly synergistically work to destroy and significantly reduce or eliminate viruses coming into contact with the hybrid antiviral metal-associated amyloid fibrils of the present disclosure. The hybrid antiviral metal-associated amyloid fibrils of the present disclosure work to synergistically treat various viruses in connection with the treatment of water, that include amyloid fibrils having one or more anti-viral metal components bound to or carried by the amyloid fibrils.

It is presently believed that the network of hybrid antiviral metal-associated amyloid fibrils of the present disclosure trap and/or cause the viruses passing through or coming into contact with the hybrid antiviral metal-associated amyloid fibrils to be killed or otherwise eliminated/inactivated viruses. The amount of virus is dramatically reduced and, in many instances, lowered to non-detectable levels, which are levels not achieved by the use of amyloid fibrils or the metal particles alone. It is specifically contemplated that the antiviral metal-associated hybrid amyloid fibrils of the present disclosure can be incorporated into each of the following: (1) face masks for general, medical, or dental use; (2) air filters for purification of any enclosed structure or space such as a room, building or vehicle; and (3) water filters or water filtration systems to eliminate or capture viruses and bacteria within the water passing through the water filter and coming into contact with the hybrid antiviral metal-associated amyloid fibrils within the water filter.

The virucidal hybrid antiviral metal-associated amyloid fibrils material of the present disclosure is typically a metal-associated hybrid amyloid fibril material usually composed of food-grade milk protein fibrils modified on their surface by metal particles, typically metal nanoparticles, more typically, iron oxide nanoparticles, silver nanoparticles, or iron hydroxide nanoparticles. As shown in at least FIGS. 2A-4 and discussed herein, the metal-associated hybrid amyloid fibrils of the present disclosure can effectively inactivate the enveloped viruses such as HBV, HCV, HIV, influenza and coronaviruses. Protein fibrils play a major role in binding strongly to the virus to the outer lipid membrane, leading to multivalent binding with the consequent inactivation of the viruses. It has been surprisingly found that while the use of protein fibrils or metal nanoparticles do not have any significant virucidal properties but the hybrid metal and amyloid material surprisingly generates a synergetic effect leading to outstanding virucidal properties when the hybrid metal and amyloid material comes into contact with a virus. Due to the challenges and biosafety concerns of working with the influenza virus and coronaviruses, the studies employed in the case of the present disclosure included testing on an enveloped bacteriophage Phi6, which has been accepted by the research community as a surrogate for the enveloped viruses (influenza virus and coronaviruses). Initially, these hybrid materials were tested for enveloped phi6—bacteriophages and then for Influenza viruses. The hybrid material of various concentrations of 2.6 mg/ml was incubated to the virus concentration range of 10⁴-10³ pfu/ml; all the phi6 and influenza virus were inactivated by 100%. In addition, the hybrid material containing membranes may be prepared by vacuum filtration and filtered a solution of virus (10³ pfu/ml). Complete purification of water such that contaminants were not detectable pfu in filtrate, which illustrates that this membrane can be used for outstanding water filtration from viruses. Since these are made with food grade materials, these antiviral material and membrane are environmental-friendly.

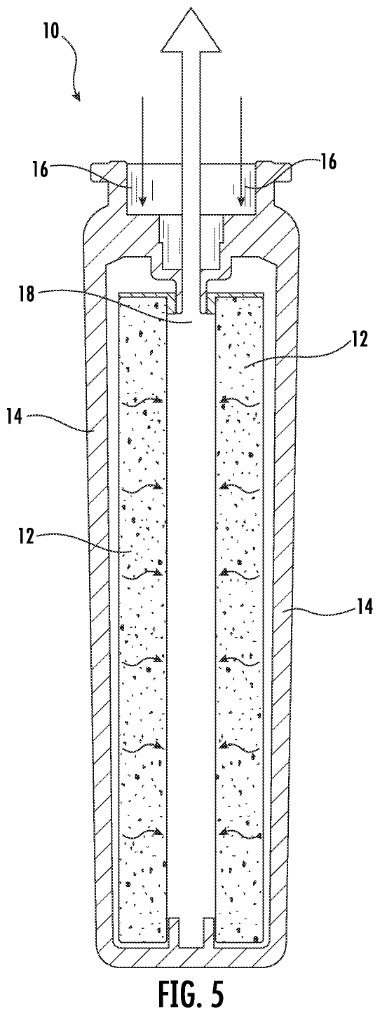

While discussed in more detail below, amyloid fibrils may be produced by heating the 2 wt. % of purified beta-lactoglobulin (BLG) protein monomer (pH 2) at 90° C. for 5 h. (scale bar is 100 nm). It is presently believed that various metal particles may be bound or otherwise associated with the amyloid fibrils, but the metal particles are more typically, nanoparticles and most typically, iron nanoparticles. Silver nanoparticles, and iron nanoparticles, typically iron hydroxide or iron oxide nanoparticles, have each been found to be particularly effective to reduce significantly viral presence when used in association with amyloid fibrils. Iron hydroxide is more sustainable food-grade material compared to other nanoparticles discussed herein. A blend of different nanoparticles could also be used and a mix of different metal particles associated with and/or bonded to the amyloid fibrils to create the hybrid antiviral metal-associated amyloid fibrils material of the present disclosure. The antiviral metal-associated amyloid fibril material effectively inactivates viruses, in particular enveloped viruses such as HBV, HCV, HIV, influenza. It is presently believed that the protein fibrils play a major role in binding strongly to the virus to the outer lipid membrane, leading to multivalent binding with the consequent inactivation of the virus. As shown in FIGS. 2A-C, FIG. 3 and FIG. 5, protein fibrils or iron hydroxide nanoparticles alone do not have significant virucidal properties, but the hybrid antiviral metal-associated amyloid fibrils material of the present disclosure generates a synergistic effect leading to outstanding virucidal properties.

Iron nanoparticles may be synthesized onto amyloid fibrils by in situ chemical reduction of FeCl₃·6H₂0 to obtain iron β-Lactoglobulin fibrils (i.e. the composite materials comprising amyloid fibrils and nanoparticulate mineral compounds located on the surface of said amyloid fibrils). 0.45 wt. % of amyloid fibrils may be mixed with 0.015M FeCl₃·6H₂0 salt solutions. Iron III ions binding to amyloid fibrils are then chemically reduced by NaBH₄ and/or NaOH. (scale bar is 100 nm). In more general terms, the present disclosure provides for the use of hybrid antiviral metal-associated amyloid fibrils having amyloid fibrils bound with antiviral metals that work synergistically to deactivate viral components where the antiviral metal is most typically an iron nanoparticle chemically engaged or bound to at least the surface of the amyloid fibrils and remain chemically engaged or bound to one another at all times while in use for antiviral treatment of a fluid, which can be air or water or any other contaminated or potentially contaminated fluid.

The hybrid antiviral metal-associated amyloid fibrils of the present disclosure are most often associated with an inedible substrate or membrane that serves to hold the hybrid antiviral metal-associated amyloid fibrils while in use during filtration. The hybrid antiviral metal-associated amyloid fibrils may be placed as a filtration media in a water filter, and air filter, or potentially a facemask or ventilator.

As discussed above the hybrid antiviral metal-associated amyloid fibrils of the present disclosure typically contain both amyloid fibrils and one or more nanoparticulate minerals located on the surface of said amyloid fibrils. The nanoparticles may be the same or a combination of different nanoparticles.

Amyloid Fibrils

The term “amyloid fibrils” is generally known in the field to describe a specific type of protein aggregates and particularly describes fibrils made by proteins or peptides prevalently found in beta-sheet secondary structure. Accordingly, the term amyloid fibrils exclude native proteins. Particularly suitable amyloid fibrils used to produce the hybrid antiviral metal-associated amyloid fibrils of the present disclosure are amyloid fibrils from β-lactoglobulin, an inexpensive milk protein with natural reducing effects, which proves to act as an anti-oxidizing nanocarrier and colloidal stabilizer for nanoparticulate minerals, such as nanoparticulate iron compounds. The amyloid fibrils are typically obtained from globular proteins, most typically food grade globular proteins. Some globular proteins that may be used to form the amyloid fibrils of the present disclosure may be from any one or combination of the following globular proteins: beta-lactoglobulin (BLG), whey, lysozyme, bovine serum albumin, soy proteins, ovalbumin and any combination thereof. However, beta-lactoglobulin is most typically preferred. It is most typically preferred because of inexpensive, readily available and highly robustness to form the amyloid fibrils. Positive charge groups available on the surface of the beta-lactoglobulin fibrils will bind the iron ions and in situ synthesis of the nanoparticles. Milk-based β-lactoglobulin fibrils are easily obtained by heating 2 wt. % β-lactoglobulin monomer solution (pH=2) at 90° C. for 5 h. These amyloid fibrils serve as a scaffold for the final hybrid materials of interest to the present proposal. In short, the solution of protein fibrils coated by iron hydroxide nanoparticles can be obtained via a simple in situ binding process by initial mixing of amyloid fibrils solution with an aqueous solution of FeCl₃·6H₂O. The nucleation and growth of the iron hydroxide nanoparticles onto the amyloid fibrils surface is achieved by a simple change in pH, allowing the conversion of iron ion precursors into the strongly bound nanoparticles. These hybrid protein fibrils show the outstanding antiviral capability when tested in the water against several enveloped viruses.

Advantageously, the amyloid fibrils typically have a high aspect ratio, preferably with 10 nm in diameter and ≥1 μm in length. The amyloid fibrils have a highly charged surface. The term highly charged surfaces are generally known in the field and particularly describes surfaces showing electrophoretic mobility of the order 2 μm·cm/V·s at pH 2. Accordingly, amyloid fibrils having electrophoretic mobility of the order 1-4 μm·cm/V·s at pH 2 are preferred. Amyloid fibrils that may be used in the context of the present disclosure include, but are not limited to, the amyloid fibrils disclosed in US Patent Application Publication No. 2017/0096349 A1, the entire disclosure of which is hereby incorporated by reference herein.

In the case of iron nanoparticles whether from iron elementally, iron hydroxide or iron oxide, it is presently believed that the resulting composite material forms a stable protein-iron colloidal dispersion. Importantly, the iron nanoparticle and amyloid composites show high antiviral capability. Formation of the composites from iron hydroxide and iron oxide may be formed in the manner described below.

Iron oxide particles in the case of the present disclosure may be produced by mixing the iron chloride FeCl₃ dissolved salt solution to the amyloid fibrils, further iron ions converted to iron oxide nanoparticles by the Sodium Borohydride (NaBH₄). NaOH adjusts the pH to pH7 for the virus incubation studies.

Silver nanoparticles may be prepared by the mixing of the silver nitrate (AgNo₃) salt solution to the amyloid fibrils. Further silver ions converted to silver nanoparticles by the Sodium Borohydride (NaBH₄). NaOH adjusts the pH to pH7 for the virus incubation studies.

Nanoparticulate Minerals

Nanoparticulate minerals are defined both by particle size and chemical composition of the particles. As the term “nano” implies, particles of 5-100 nm size (as determined by microscopy) are particularly useful in the context of the present disclosure. The particle size may vary, depending on the mineral. As an exemplary embodiment, for iron, a preferred range of the particles is from about five to about 20 nm. As a further exemplary embodiment, for iron and/or silver, a typical range of the particles is from about 20 to about 100 nm.

As discussed herein, iron oxide and iron hydroxide and silver nanoparticles have all been shown to synergistically dramatically reduce and eliminate to an undetectable level of various bacteria and viruses.

Broadly speaking, it is believed that any known metal may be used; preferred are minerals as defined above. As used herein, the term minerals shall particularly include compounds selected from the group consisting of salts, oxides and hydroxides. Other metal components applicant presently believes will also exhibit synergistic antiviral properties in addition to iron include silver, gold, copper, titanium, platinum, nickel, aluminum and palladium.

The metal nanoparticles are predominantly located on the surface of the amyloid fibrils, such as at least 80%, more typically, at least 90% of the nanoparticles, and most typically at least 95% of the nanoparticles are located on the surface (with respect to the total amount of nanoparticles present in the composite). Particularly preferably, all nanoparticles are present on the surface of the amyloid fibrils. It was surprisingly found that the amyloid fibrils and metal nanoparticles of the composite material synergistically interact to eliminate viruses in vitro. The ratio of both constituents may vary over a broad range, depending inter alia on the specific materials and the intended use. Particularly preferred hybrid antiviral metal-associated amyloid fibrils are obtained when the ratio of amyloid fibrils to metal nanoparticles is in the range of from about 20/1 to about 1/1 (w/w), such as about 5/1.

An enveloped virus has an outer lipid layer of glycoprotein and lipoproteins. Many enveloped viruses, such as HBV, HCV, HIV, influenza, and coronaviruses, are pathogenic to humans and of clinical importance. Infectious diseases caused by enveloped viruses, such as influenza viruses and the coronaviruses are responsible for severe acute respiratory syndrome (SARS) and the Middle East respiratory syndrome (MERS), cause thousands of deaths and billions of dollars of economic losses per year. After infection, antiviral drugs are the only treatment option presently available and these are only limited to antiviral drugs that have received regulatory approval in a given country or jurisdiction, which are often quite limited or non-existent for certain viruses. The hybrid antiviral metal-associated amyloid fibrils of the present disclosure typically include a non-toxic food grade protein fibril component containing iron or silver particles, typically nanoparticles, thereon. The metal particles, which are typically iron or silver nanoparticles act as at least a moderate virucidal or a virucidal for a broad spectrum of enveloped viruses. Furthermore, the same materials can be used as membranes for virus filtration for dangerous waterborne viruses and diseases, including meningitis, hepatitis, gastroenteritis, conjunctivitis, SARs, hepatitis A and E viruses. Current membrane filtration methods require intensive energy to remove pathogenic viruses adequately. The membranes with hybrid antiviral metal-associated amyloid fibrils of the present disclosure can safely and effectively remove viruses from drinking water. The membranes with hybrid antiviral metal-associated amyloid fibrils of the present disclosure are also presently believed to function to safely and effectively remove viruses from the air or aerosol droplets in the air.

As discussed above, the virucidal hybrid antiviral metal-associated amyloid fibrils of the present disclosure typically include the synergistic combination of food-grade milk protein fibrils modified on their surface by iron hydroxide nanoparticles. This material can effectively inactivate the enveloped virus such as HBV, HCV, HIV, influenza and coronaviruses, such as COVID-19. The protein fibrils play a major role in binding strongly to the virus to the outer lipid membrane of a virus. This leads to multivalent binding with the consequent inactivation of the virus. Protein fibrils, silver, iron oxide, or iron hydroxide nanoparticle alone simply do not have any virucidal properties or at least any significant virucidal properties, but the hybrid antiviral metal-associated amyloid fibrils surprisingly generate a synergetic effect leading to outstanding virucidal properties.

Since the water and/or air filtration products incorporating the hybrid antiviral metal-associated amyloid fibrils of the present disclosure are made with food-grade materials, these antiviral materials and membrane are environmentally friendly and would decompose within two years. The hybrid antiviral metal-associated amyloid fibrils of the present disclosure act as virucidal for a broad spectrum of enveloped viruses.

Viruses commonly occur in drinking water sources, and certain types (e.g., adenoviruses, rotaviruses) are resistant to several traditional and alternative disinfection techniques, such as monochloramination and UV254 irradiation. While free chlorine is generally highly effective for virus inactivation, its use is limited due to the potential to form toxic disinfection byproducts (DBPs). Current membrane filtration methods require intensive energy to remove pathogenic viruses adequately without using chemicals like chlorine. Amyloid iron hydroxide composite membranes can be used effectively and safely for the filtration of viruses from drinking water.

This material has the potential to effectively inactivating the enveloped virus such as HBV, HCV, HIV, influenza and various coronaviruses such as COVID-19. Protein fibrils play a major role in binding strongly to the virus to the outer lipid membrane, leading to multivalent binding with the consequent inactivation of the virus. Protein fibrils or iron hydroxide nanoparticles alone do not have any virucidal properties but the hybrid material generates a synergistic effect leading to outstanding virucidal properties.

In order to demonstrate the feasibility of the approach, the enveloped bacteriophage Phi6, which has been suggested as a surrogate for the influenza virus and SARS coronavirus, as well as Influenza A viruses were tested. Hybrid material was incubated in the water at a concentration of 2.6 mg/ml to the virus concentration range of 10⁴-10⁷ pfu/ml; all the phi6 and influenza virus were inactivated by 100% (n.d.=not detectable). The virus was also found to be completely removed from water filtered through the membranes made of amyloids modified by iron-hydroxide nanoparticles, but no inactivation was found in any of the control experiments. This demonstrated that the β-lactoglobulin amyloid fibril-iron hydroxide nanoparticle hybrids have the potential to serve as antiviral filters when used to prepare membranes made thereof (See FIG. 2).

Water Filters

The virucidal hybrid antiviral metal-associated amyloid fibrils material of the present disclosure may be one of or the only filtration medium using in any type of water filter, but can also be one stage or comingled with another water filtration medium such as activated carbon particles. FIG. 5 shows one type of water filter 10 where water filtration material 12 is contained within a housing 14. The housing has a water inlet 16 that receives water to be treated by the water filter and a treated water outlet 18. Water filters may be pressurized water filters or gravity fed water filters. The nature of the water filter construction is not presently believed to be critical so long as at least one of the filter materials used in association with the water filter is the virucidal hybrid antiviral metal-associated amyloid fibril material of the present disclosure. One such gravity fed filter that may be used is a BRITA® water filter. Additionally, the virucidal hybrid antiviral metal-associated amyloid fibril material of the present disclosure may be placed on or woven into or otherwise associated with a support material such as a cellulosic membrane. Support materials may or may not be present. For many applications, such support materials are preferred and may be selected from a broad range of known materials. The choice of support material depends on its intended use. Suitable support materials are, for example, porous support materials. In certain applications, it is advantageous the support material being a carbonaceous material that readily oxidizes in a furnace, such as cellulose membranes. Composites of the virucidal hybrid antiviral metal-associated amyloid fibril material of the present disclosure and a membrane or other support material may be used for the purification of any fluid, either a liquid or of air contaminated by COVID-19 and/or other respiratory viruses. Significantly, the support material, while typically an organic material, in particular an organic cellulosic material, the support material used may be an inorganic material or a blend of organic and inorganic materials. The virucidal hybrid antiviral metal-associated amyloid fibril material of the present disclosure may be placed on the support material as a distinct layer or conceivably mixed into the support material such that it is comingled or at least partially or completely resides within the support material. The virucidal hybrid antiviral metal-associated amyloid fibril material of the present disclosure may also be layered on and spaced within the support material.

Air Filter(s) and Facemasks

The hybrid antiviral metal-associated amyloid fibrils may be incorporated into any of a variety of facemasks and/or purifying membranes (ventilator filter units) for circulation of air in clinics or hospitals treating infected patients. The virucidal hybrid antiviral metal-associated amyloid fibril material of the present disclosure may also be incorporated within or as a layer on the surface of an air filter or facemask or a combination of on the surface or within the air filter or facemask. The air filter may be an air filter associated with a heating and/or cooling air conditioning system or simply an airflow system. The air filter could also conceivably be used in connection with any air-moving device for filtering air within a single room as part of a stand-alone air filter system. The air moving device or heat and cooling or other air treatment system typically includes a fan operably connected to a motor, which is typically an electrical motor, such that the fan moves its blades to push air through the air filter or pull air through the air filter such that air or aerosols (a suspension of fine solid particles or liquid droplets in air or another gas) containing virus, bacteria or other contaminant is contacted with the virucidal hybrid antiviral metal-associated amyloid fibril material. In any case, air is caused to flow into contact with an air filter for any defined interior volume within a building structure or a vehicle or a portion thereof. The heat and/or cooling or other air treatment systems within a building are associated with a series of ductwork to deliver conditioned air to one or more and typically a plurality of rooms or regions within the home in order to deliver filtered and typically also conditioned air to the regions. Air flows through the air filter or a series of air filters of the present disclosure such that viral and/or bacterial components and other impurities are removed. The air filters of the present disclosure not only include a layer or other configuration of virucidal hybrid antiviral metal-associated amyloid fibril material of the present disclosure, but may also include other air filtration components such as a spun fiberglass material or a pleated paper or cloth enclosed in a frame. The other air filtration components such as spun fiberglass material or paper cloth or other filter material may be entirely independent of the virucidal hybrid antiviral metal-associated amyloid fibril material of the present disclosure or these materials may serve as a substrate or carrier for the virucidal hybrid antiviral metal-associated amyloid fibril material of the present disclosure. The air filter of the present disclosure may incorporate a prefilter that filters larger contaminants. Another filter material that may be incorporated into the overall filter assemblies of the present disclosure may include borosilicate glass fibers or plastic fibers (e.g., polypropylene) bound together with up to 5% acrylic binder. An electrostatic precipitator may also be included into the overall assembly. The electrostatic precipitator, if used, generates ions by running extremely high positive direct current voltages through steel wires set between grounded steel charging plates. The housing for an air filter assembly of the present disclosure may include a hard housing or be constructed of cardboard or other paper product(s). When a hard housing is employed, the housing is typically made from plastic, usually high-impact polystyrene, polyvinyl chloride, high-density polyethylene, or polypropylene. Most air filter systems/assemblies are also usually equipped with a post-filter composed of activated carbon. The virucidal hybrid antiviral metal-associated amyloid fibril material of the present disclosure may be associated with and/or a layer of any part of the filter assembly. It may also be an independent material layer alone or on a substrate or membrane and incorporated into the overall assembly. The virucidal hybrid antiviral metal-associated amyloid fibril material of the present disclosure may also be a part of or layer on the post-filter alone instead of activated carbon or in addition to the activated carbon typically included in the post filter.

In the case of a facemask for use by any person, but in particular for potential use by veterinary, dental and health professionals, the facemask is typically a woven or unwoven fabric material with the virucidal hybrid antiviral metal-associated amyloid fibril material of the present disclosure within the fabric material, on the fabric material as a layer or both. Nonwoven fabrics are broadly defined as sheet or web structures bonded together by entangling fiber or filaments (and by perforating films) mechanically, thermally or chemically. Woven fabrics are typically made by using two or more sets of yarn, typically a naturally occurring, but possibly a synthetic yarn, interlaced at right angles to each other. Weaving produces a variety of types of material. Woven fabrics are generally very durable. Woven fabrics can be easily cut into different shapes and are excellent for producing styles in garments. The raw edges of woven fabrics ravel or fray easily and often need to be protected. Fabrics having more fabric count (number of wrap and weft yearns present) keep their shape well. Low count fabrics are less durable and may snag or stretch. Woven fabrics are manufactured in different widths depending on the end-use. The fabrics used for apparels usually contain 90 cm in width. The Sheeting materials are generally made having a width of from about 160 cm to about 140 cm and about 150 cm to 180 cm.

By contrast, as discussed above already, nonwovens do not depend on the interlacing of yarn for internal cohesion. Nonwovens typically do not have an organized geometrical structure. They are essentially the result of the relationship between one single fiber and another. This provides nonwoven fabrics with characteristics of their own, with new or better properties such as absorption and filtration and therefore opens them up to other applications.

The present disclosure is generally directed to an antiviral material prepared by non-toxic food grade protein fibrillary material modified with iron hydroxide, iron oxide, or silver nanoparticles and/or combinations thereof. These materials act as virucidal for a broad spectrum of enveloped viruses. Since this technology is scalable, inexpensive, non-toxic (the precursor materials are food-grade), sustainable, ease of implementation, multifunctional, ideally effective for a broad range of enveloped viruses, this technology could become directly applicable as a filtration device for the prevention of the COVID-19 virus and similar respiratory viruses.

The present disclosure also incorporates the virucidal hybrid antiviral metal-associated amyloid fibril material of the present disclosure in connection with respirators and surgical masks typically denoted with names such as N95 masks or personal protective equipment.

A surgical mask typically refers to a loose-fitting, disposable device that creates a physical barrier between the mouth and nose of the wearer and potential contaminants in the immediate environment. Surgical masks are regulated under 21 CFR § 878.4040. Surgical masks are not shared and may be labeled as surgical, isolation, dental, or medical procedure masks. They may come with or without a face shield. These are often referred to as facemasks, although not all face masks are regulated as surgical masks.

An N95 respirator is a respiratory protective device designed to achieve a very close facial fit and very efficient filtration of airborne particles. Edges of the respirator are designed to form a seal around the nose and mouth. Surgical N95 respirators are commonly used in healthcare settings and are a subset of N95 Filtering Facepiece Respirators (FFRs), often referred to as N95 facemasks. N95 facemasks filter a minimum of 95% filtration efficiency against solid and liquid aerosols that do not contain oil and may or may not be resistant to synthetic blood directed at it under high pressures (see ASTM F1862, which is a standard test method for resistance of medical facemasks to penetration by synthetic blood. This test is required because during certain medical procedures, a blood vessel may occasionally be punctured, resulting in a high-velocity stream of blood impacting a protective medical facemask. The test procedure specifies that a mask or respirator is conditioned in a high-humidity environment to simulate human use and is placed on a test holder. Synthetic blood (2 cc) is shot horizontally at the mask at a distance of 30 cm (12 inches). Surgical masks and respirators are tested on a pass/fail basis at three velocities corresponding to the range of human blood pressure (80, 120, and 160 mm Hg). The inside of the mask is then inspected to see if any synthetic blood has penetrated to the inside of the facemask. Fluid resistance according to this test method is when the device passes at any level.

In the case of the present disclosure, all of the facemasks and respirators typically used in connection with any healthcare or veterinary or dental personal safety device may incorporate a layer or elements that include the virucidal hybrid antiviral metal-associated amyloid fibril material of the present disclosure. As such, the facemasks and respirators protect the wearer from viral and bacterial components that may come into contact with the virucidal hybrid antiviral metal-associated amyloid fibril material of the present disclosure in connection with the use of the facemask or other personal protection device. The viral and bacterial components are captured, eliminated, and/or killed such that the wearer of the personal protection device avoids infection. The masks and other personal protective devices of the present disclosure also typically include one, but more typically two elastic bands affixed to opposite sides of the mask to attach the personal protective device to the wearer, usually by wrapping the strap over the ears or around the back of the head (especially if the mask is to be worn for an extended period of time so as to avoid irritation of the wearer's ears).

1. A water or air filtration apparatus comprising a support material that holds on or within it an antiviral composition comprising metal nanoparticles on a surface of an amyloid fibril thereby forming a hybrid antiviral metal-associated amyloid fibril and wherein the metal nanoparticles are a metal nanoparticle chosen from the group consisting of a silver nanoparticle, an iron hydroxide nanoparticle, an iron oxide nanoparticle and mixtures thereof.

2. The water or air filtration apparatus of claim 1, wherein the amyloid fibril is an amyloid fibril chosen from the group consisting of: a beta-lactoglobulin, whey, lysozyme, bovine serum albumin, soy proteins, ovalbumin, and mixtures thereof.

3. The water or air filtration apparatus of claim 1, wherein the amyloid fibril is a milk based β-lactoglobulin fibril.

4. (canceled)

5. The water or air filtration apparatus of claim 3, wherein the support material is a membrane material and is an organic material or a combination of an organic and an inorganic material.

6. The water or air filtration apparatus of claim 1, wherein the support material is chosen from the group consisting of a cellulosic, activated carbon material and a fabric material.

7. The water or air filtration apparatus of claim 1, wherein the metal nanoparticles have a particle size of from about 5 nm to about 900 nm.

8. The water or air filtration apparatus of claim 7, wherein the metal nanoparticles have a particle size of from about 20 nm to about 100 nm.

9. The water or air filtration apparatus of claim 1, wherein the metal nanoparticles are chemically engaged or bound to at least the surface of the amyloid fibrils.

10. The water or air filtration apparatus of claim 9, wherein the metal nanoparticles are chemically engaged or bound to at least the surface of the amyloid fibrils and the metal nanoparticles remain chemically engaged or bound to one another at all times while in use for antiviral treatment of a fluid.

11. The water or air filtration apparatus of claim 10, wherein the fluid is ambient air containing one or more ambient air contaminants or water containing one or more water contaminants.

12. The water or air filtration apparatus of claim 1, wherein at least 80% of the metal nanoparticles are present on the surface of the amyloid fibril.

13. The water or air filtration apparatus of claim 1, wherein the hybrid antiviral metal-associated amyloid fibril is spaced within a housing of a water or air filter having a water inlet or an air inlet and a water outlet or an air outlet and a water flow path or an air flow path that requires at least a portion of the water or air coming into the water inlet to pass through or into contact with the hybrid antiviral metal-associated amyloid fibril prior to exiting the water outlet or the air outlet.

14. The water or air filtration apparatus of claim 11, wherein the support material is a woven or non-woven material and the hybrid antiviral metal-associated amyloid fibril is on the surface of, spaced within or both on the surface of or spaced within the woven or non-woven material and wherein the woven or non-woven material is sized to cover at least a human mouth and nose.

15. The water or air filtration apparatus of claim 14, wherein the woven or non-woven material has at least one elastic member bound thereto such that the at least one elastic member provides a force to retain the woven or non-woven mask over a wearer's nose and mouth when placed over the wearer's nose and mouth.

16. The water or air filtration apparatus of claim 1, wherein the water or air filtration apparatus removes viral and bacterial contaminants to non-detectable levels.

17. The water or air filtration apparatus of claim 16, wherein the water or air filtration apparatus is chosen from the group consisting of a mask, a water filtration cartridge, a whole home air filter, and an air filter for a ventilator.

18. The water or air filtration apparatus of claim 1, wherein the hybrid antiviral metal-associated amyloid fibril is a component of a personal protective mask to cover at least a wearer's mouth and nose.

19-20. (canceled)

21. A filtration apparatus comprising an antiviral composition comprising metal nanoparticles on a surface of an amyloid fibril and a housing that contains the antiviral composition and wherein the antiviral composition has synergistic antiviral properties compared to the amyloid fibrils alone and the antiviral composition removes virus and bacterial contaminants in water or air to non-detectable levels when water or air comes into contact with the antiviral composition;
wherein the amyloid fibril is an amyloid fibril chosen from the group consisting of: a beta-lactoglobulin, whey, lysozyme, bovine serum albumin, soy proteins, ovalbumin and mixtures thereof; and
wherein the metal nanoparticles have a particle size of from about 5 nm to about 900 nm and are chosen from the group consisting of a silver nanoparticle, an iron hydroxide nanoparticle, an iron oxide nanoparticle and mixtures thereof.

22-121. (canceled)

122. A heating, ventilation, and air conditioning system associated with a vehicle, an interior volume of a structure, or a portion of the interior volume of a structure, the heating, ventilation, and air conditioning system comprising: a water filtration apparatus or air filtration apparatus comprising a support material holding an antiviral composition on or within the support material, the antiviral composition comprising metal nanoparticles on a surface of an amyloid fibril thereby forming a hybrid antiviral metal-associated amyloid fibril and wherein the metal nanoparticles are a metal nanoparticle chosen from the group consisting of a silver nanoparticle, an iron hydroxide nanoparticle, an iron oxide nanoparticle and mixtures thereof.

123. The heating, ventilation, and air conditioning system associated with a vehicle, an interior volume of a structure, or a portion of the interior volume of a structure of claim 122, wherein the amyloid fibril is an amyloid fibril chosen from the group consisting of: a beta-lactoglobulin, whey, lysozyme, bovine serum albumin, soy proteins, ovalbumin and mixtures thereof; and the metal nanoparticles have a particle size of from about 5 nm to about 900 nm.
